(19) United States
(12) Patent Application Publication
(10) Pub. No.: US 2012/0076736 A1
(43) Pub. Date: Mar. 29, 2012

(54) HOST SUPPORTED GENETIC BIOSENSORS

(75) Inventor: David A. Borkholder, Canandaigua, NY (US)

(73) Assignee: ROCHESTER INSTITUTE OF TECHNOLOGY, Rochester, NY (US)

(21) Appl. No.: 13/234,992

(22) Filed: Sep. 16, 2011

Related U.S. Application Data
(60) Provisional application No. 61/384,098, filed on Sep. 17, 2010.

(51) Int. Cl. A61K 49/00

(52) U.S. Cl. 424/9.6; 424/9.1

(57) ABSTRACT

The present invention relates to an in vivo method of monitoring an analyte in a subject. This method involves providing an expression vector that encodes a biosensor molecule, the biosensor molecule comprising an analyte binding domain and a signal domain. The biosensor molecule produces a signal from the signal domain upon binding of the analyte by the analyte binding domain. The signal is detectable by a non-invasive means. The expression vector is introduced locally into in vivo cells of a subject under conditions effective to express the biosensor molecule in the cells. The signal from the expressed biosensor molecule is detected by a non-invasive means, thereby monitoring the analyte in the subject in vivo.

This application claims the benefit of U.S. Provisional Patent Application Ser. No. 61/384,098, filed Sep. 17, 2010, which is hereby incorporated by reference in its entirety.

### FIELD OF THE INVENTION

This invention generally relates to host supported genetic biosensors that can be used for non-invasive detection of a physiological molecule or analyte and to methods which employ such biosensors.

### BACKGROUND OF THE INVENTION

Diabetes remains a significant health risk for the American population. In 2007, almost 24 million people were impacted, representing 8% of the population. Those over the age of 60 are at higher risk, with disease prevalence exceeding 23% (“National Diabetes Fact Sheet: General Information and National Estimates on Diabetes in the United States,” Centers for Disease Control (2007); U.S. Department of Health and Human Services, Centers for Disease Control and Prevention (2008)). These statistics, coupled with the high prevalence of obesity and the aging population in the U.S. suggest effective management of diabetes will become increasingly important in the future. Effective control of diabetes requires monitoring of blood glucose levels, which is generally accomplished via a blood sample taken from the patient. This method, while effective, is painful and does not allow continuous monitoring throughout the day.

Determining physiological levels of molecules or analytes (e.g., glucose, vitamins, biomarkers, signaling molecules, therapeutic drugs, hormones) normally entails withdrawing a blood sample from a patient, and then analyzing the sample in vitro. This approach has limitations that may include high cost, time delay in obtaining results, patient discomfort and inconvenience associated with periodic blood draws, and testing prerequisites such as fasting. There is a need for methods which overcome these limitations and allow for continuous analyte measurement, which would lead to more convenient and better monitoring of several human disorders and drug treatments. For example, if patients with diabetes were able to continuously monitor a display of glucose concentration in blood or tissue, they could better avoid extremes of glycemia and reduce their risk for long term complications.

One approach to continuous monitoring is by implanting biosensors or medical devices, such as glucose monitors, into the patient. Implanted devices are impacted by the host system, with inflammation and fibrosis around the implant, thus degrading sensor performance (Moussy, Sensors 1:270-273 (2002)). Fibrosis of the foreign body typically results in development of a capsule around the implanted sensors 3 to 4 weeks after implantation and can reduce the influx of substrates such as glucose and oxygen (Ward et al., ASAIO J. 45:555-561 (1999); Updike et al., Diabetes Care 23:208-214 (2000); Gilligan et al., Diabetes Care 17:882-7 (1994)). Further, problems associated with energy use, efficient functioning, and life span of the implanted biosensors is a big impediment in the development and usage of such implantable biosensors.

The fundamental task required of implantable biosensors or medical devices is accurate real-time determination of relevant functional physiological molecules or analytes. Nonetheless, typically, in vivo biosensors only approximate physiological function via the measurement of surrogate signals and so are prone to introduction of error in biological monitoring (Celiker et al., Pacing Clin. Electrophysiol. 21:2100-2104 (1998)). Electrochemical enzyme sensors (e.g., glucose oxidase) are prone to fouling in both implanted and externally worn devices. External devices suffer from poor access to interstitial fluid with widely varying measurement success. For these reasons, both external and implantable biosensors have not gained widespread acceptance. There exists a need for an entirely new approach to in vivo analyte monitoring which provides the sensitivity of an implanted system while avoiding the issues commonly associated with both external and implanted devices.

The present invention is directed to overcoming these and other deficiencies in the art.

### SUMMARY OF THE INVENTION

A first aspect of the present invention relates to an in vivo method of monitoring an analyte in a subject. This method involves providing an expression vector that encodes a biosensor molecule, the biosensor molecule comprising an analyte binding domain and a signal domain. The biosensor molecule produces a signal from the signal domain upon binding of the analyte by the analyte binding domain. The signal is detectable by a non-invasive means. The expression vector is introduced locally into in vivo cells of a subject under conditions effective to express the biosensor molecule in the cells. The signal from the expressed biosensor molecule is detected by a non-invasive means, thereby monitoring the analyte in the subject in vivo.

The present invention relates to molecular scale protein biosensors which offer new opportunities for continuous analyte detection. The methods described herein are extendable for detection of a diverse set of physiological molecules and analytes.

Biosensor molecules of the present invention are genetically expressed locally (as opposed to systemically) in in vivo cells of a subject. Biosensor molecules are capable of binding to a physiological molecule or analyte of interest in the subject. Such binding produces a biological signal that can be detected (monitored) and correlated with the amount of the physiological molecule or analyte present in the subject.

While the past several decades have witnessed considerable focus on development of both implantable and external devices for continuous analyte (e.g., glucose) monitoring, sensor lifetimes continue to be an issue. The genetically expressed biosensors described in the present invention offer a new approach to continuous detection of physiological molecules and analytes at localized locations in a subject. This will lead to efficient detection, prevention, and better management of a variety of biological disorders by providing continous physiological feedback via a non-invasive detection means.

### BRIEF DESCRIPTION OF THE DRAWINGS

FIG. 1 is a schematic illustration showing Förster resonance energy transfer (“FRET”) protein expression in the epithelial layer of a subject, which allows optical detection of glucose concentration in localized interstitial fluid. CFP=Cyan Fluorescent Protein, YFP=Yellow Fluorescent Protein.

FIG. 2 is a schematic illustration of one embodiment of the present invention. An expression vector is introduced locally into in vivo cells of animal model 100. Localized in vivo cells 106 of animal model 100 express the biosensor molecule of the present invention. In vivo cells 106 are then exposed to a specific wavelength of incident light using light source 102. Upon interaction with the incident light the biosensor molecule, expressed in in vivo cells 106, exhibits a fluorescent signal which is emitted and can be detected and quantified using detector 104.

### DETAILED DESCRIPTION OF THE INVENTION

A first aspect of the present invention relates to an in vivo method of monitoring an analyte in a subject. This method involves providing an expression vector that encodes a biosensor molecule, the biosensor molecule comprising an analyte binding domain and a signal domain. The biosensor molecule produces a signal from the signal domain upon binding of the analyte by the analyte binding domain. The signal is detectable by a non-invasive means. The expression vector is introduced locally into in vivo cells of a subject under conditions effective to express the biosensor molecule in the cells. The signal from the expressed biosensor molecule is detected by a non-invasive means, thereby monitoring the analyte in the subject in vivo.

Biosensor molecules suitable for use in the method of the present invention have at least one analyte binding domain and at least one signal domain. The analyte binding domain binds a target analyte present in a subject. The analyte binding domain can be engineered for binding efficacy. For example, a glucose/galactose-binding protein can be mutated to alter glucose binding affinity. Usually, the analyte binding domain is engineered to enhance binding efficacy, but sometimes it may be desirable to engineer the binding domain so that its binding efficacy is diminished. This would typically be the case when the signal emanating from the biosensor molecule is so high that it cannot be effectively measured.

In one embodiment of the present invention, binding of the analyte by the analyte binding domain of the biosensor molecule causes a conformational change in the analyte binding domain. This conformational change leads to a detectable signal from the signal domain of the biosensor molecule. For example, the conformational change may cause a change in the distance between two atoms in a molecule. In another embodiment, the analyte upon binding to the analyte binding domain can cause a change in the conformation of the signal domain. The conformational change may, alternatively or in addition, also lead to activation of enzymatic activity in the analyte binding domain to convert an otherwise dormant molecule into a signaling molecule.

The signal domain of the biosensor molecule responds to binding of the analyte by the analyte binding domain by undergoing at least one biochemical or structural change. The signal domain, as referred to herein, includes signal domains which do not directly produce a detectable signal but can help or assist in producing a detectable signal. In one embodiment, the signal domain comprises a fluorescent protein domain.

The biosensor molecule can have one or multiple signal domains which are used for signal detection. Thus, in one embodiment, the biosensor molecule has a first signal domain and a second signal domain. The second signal domain can work in either the same or a different manner to that of the first signal domain, e.g., by providing an additional biochemical or structural signal to, e.g., amplify the signal obtained from the sensing domain. According to this embodiment, the second signal domain may be, e.g., a fluorescent protein domain.

In one embodiment, binding of the analyte by the analyte binding domain causes a conformational change in the signal domain. For example, the signal domain(s) may be a fluorescent protein pair which undergoes a conformational change in response to binding of the analyte, thus changing the spectral properties of the emission or changes of the emission intensity. One way of detecting such a conformational change is by the well-known method of FRET. In FRET analysis, energy transfer between two fluorophores (known as FRET probes or pairs) depends on the distance between the fluorophores. This distance between the two FRET probes changes as a result of binding of the analyte, providing a spectral shift in output fluorescence of the FRET probes. An external fluorometer can then be used to measure the relative FRET intensity at each emission wavelength, indirectly measuring the concentration of the target molecule.

A variety of FRET probes are known and used in the art. FRET probes can be characterized in cultured cell systems prior to in vivo testing. According to this embodiment of the present invention, first and second fluorescent protein domains (signal domains) are separated by the analyte binding domain. According to this arrangement, binding of the analyte by the analyte binding domain causes an increase or a decrease of the distance between two fluorescent probes or protein domains (signal domains), thus resulting in a detectable signal.

In preferred embodiments, either or both of the donor and acceptor moieties is a fluorescent protein. Suitable fluorescent proteins include green fluorescent proteins (“GFP”) (Pollock et al., Trends in Cell Biology 9:57 (1999), which is hereby incorporated by reference in its entirety), red fluorescent proteins (“RFP”), yellow fluorescent proteins (“YFP”), blue fluorescent proteins (“BFP”), and cyan fluorescent proteins (“CFP”). Useful fluorescent proteins also include mutants and spectral variants of these proteins which retain the ability to fluoresce. For example, the fluorescent proteins can include enhanced GFP, YFP, BFP, RFP, CFP, Nile Red, DsRed, T1, Dimer2, and/or mRFP1 (Shaner et al., Nature Biotechnology 22:1567 (2004), which is hereby incorporated by reference in its entirety). Mutated, modified, and other forms of fluorescent proteins are known and can be used in the method of the present invention. In addition, endogenously fluorescent proteins have been isolated and cloned from a number of marine species including the sea pansies Renilla reniformris, R. kollikeri, and R. mullerei and from the sea pens Ptilosarcus, Stylatula, and Acanthoptilum, as well as from the Pacific Northwest jellyfish, Aequorea Victoria (Prasher et al., Gene, 111:229-233 (1992), which is hereby incorporated by reference in its entirety). Also, infra red fluorescent proteins (IFPs) from bacteria (Shu et al., Science 324:804 (2009), which is hereby incorporated by reference in its entirety), and several species of coral (Matz et al. Nature Biotechnology, 17:969-973 (1999), which is hereby incorporated by reference in its entirety) are known and may be useful in the methods of the present invention.

The green-fluorescence protein, when expressed in transfected or infected cells, shines green under ultraviolet light. New versions of green fluorescent protein have been developed, such as a “humanized” GFP DNA, the protein product of which has increased synthesis in mammalian cells. One such humanized protein is “enhanced green fluorescent protein” (EGFP). Other mutations to green fluorescent protein have resulted in blue-, cyan- and yellow-green light emitting versions, and can be used in the present invention.

Biosensor molecules of the present invention are, according to one embodiment, encoded by a nucleic acid molecule (e.g., DNA) which is inserted into an expression vector. Such a nucleic acid molecule may include a gene that encodes the biosensor molecule of the present invention, which is partly or entirely heterologous (i.e., foreign) to a cell into which the expression vector is introduced. Alternatively, the nucleic acid molecule encoding the biosensor molecule is a gene homologous to an endogenous gene of the cell into which the expression vector is introduced. For example, a stem cell transformed with a vector containing an expression cassette can be used to produce a population of cells having altered phenotypic characteristics.

As used herein, an “expression vector” (sometimes referred to as gene delivery or gene transfer vehicle) refers to a macromolecule or complex of molecules comprising a polynucleotide to be delivered to a host cell, either in vitro or in vivo. The polynucleotide to be delivered may comprise a gene sequence of interest. In the present invention, the gene or nucleotide of interest encodes a biosensor molecule. Expression vectors include, for example, transposons and other site-specific mobile elements, viral vectors, e.g., adenovirus, adeno-associated virus (AAV), poxvirus, papillomavirus, lentivirus, herpesvirus, foamivirus, retrovirus vectors, pseudotyped viruses, liposomes and other lipid-containing complexes, and other macromolecular complexes (e.g., DNA coated gold particles, polymer-DNA complexes, liposome-DNA complexes, liposome-polymer-DNA complexes, virus-polymer-DNA complexes, e.g., adenovirus-polylysine-DNA complexes, and antibody-DNA complexes) capable of mediating delivery of a polynucleotide to a host cell.

Expression vectors may contain other components or functionalities that further modulate gene delivery and/or gene expression, or that otherwise provide beneficial properties to the cells in which the vectors are introduced. Such other components include, for example, components that influence binding or targeting to cells (including components that mediate cell-type or tissue-specific binding); components that influence uptake of the vector nucleic acid by the cell; components that influence localization of the polynucleotide within the cell after uptake (such as agents mediating nuclear localization); and components that influence expression of the polynucleotide. Such components also might include markers, such as detectable and/or selectable markers that can be used to detect or select for cells that have taken up and are expressing the nucleic acid delivered by the vector. Such components can be provided as a natural feature of the vector (such as the use of certain viral vectors that have components or functionalities mediating binding and uptake), or vectors can be modified to provide such functionalities. A large variety of such vectors are known in the art and are generally available. When a vector is maintained in a host cell, the vector can either be stably replicated by the cells during mitosis as an autonomous structure, incorporated within the genome of the host cell, or maintained in the host cell's nucleus or cytoplasm.

When employed, selectable markers can be positive, negative, or bifunctional. Positive selectable markers allow selection for cells carrying the marker, whereas negative selectable markers allow cells carrying the marker to be selectively eliminated. A variety of such marker genes have been described, including bifunctional (i.e., positive/negative) markers (see, e.g., WO 92/08796 and WO 94/28143, which are hereby incorporated by reference in their entirety).

In one embodiment of the present invention, the expression vector carrying the nucleic acid molecule encoding the biosensor molecule of the present invention is an expression vector derived from a virus. Suitable viral vectors include, without limitation, adenovirus, adeno-associated virus, retrovirus, lentivirus, or herpes virus.

Adenovirus viral vector gene delivery vehicles can be readily prepared and utilized as described in Berkner, Biotechniques 6:616-627 (1988); Rosenfeld et al., Science 252:431-434 (1991); WO 93/07283 to Curiel et al.; WO 93/06223 to Perricaudet et al.; and WO 93/07282 to Curiel et al., which are hereby incorporated by reference in their entirety. Adeno-associated viral gene delivery vehicles can be constructed and used to deliver a gene, including a gene encoding an antibody to cells as described in Shi et al., Cancer Res. 66:11946-53 (2006); Fukuchi et al., Neurobiol. Dis. 23:502-511 (2006); Chatterjee et al., Science 258:1485-1488 (1992); Ponnazhagan et al., J. Exp. Med. 179:733-738 (1994); and Zhou et al., Gene Ther. 3:223-229 (1996), which are hereby incorporated by reference in their entirety. In vivo use of these vehicles is described in Flotte et al., Proc. Nat'l. Acad. Sci. 90:10613-10617 (1993) and Kaplitt et al., Nature Genet. 8:148-153 (1994), which are hereby incorporated by reference in their entirety. Additional types of adenovirus vectors are described in U.S. Pat. No. 6,057,155 to Wickham et al.; U.S. Pat. No. 6,033,908 to Bout et al.; U.S. Pat. No. 6,001,557 to Wilson et al.; U.S. Pat. No. 5,994,132 to Chamberlain et al.; U.S. Pat. No. 5,981,225 to Kochanek et al.; U.S. Pat. No. 5,885,808 to Spooner et al.; and U.S. Pat. No. 5,871,727 to Curiel, all of which are hereby incorporated by reference in their entirety.

Retroviral vectors that have been modified to form infective transformation systems can also be used to deliver nucleic acid molecules encoding a biosensor molecule. One such type of retroviral vector is disclosed in U.S. Pat. No. 5,849,586 to Kriegler et al., which is hereby incorporated by reference in its entirety.

Pursuant to the method of the present invention, the expression vector of the present invention is transcribed and, optionally, translated into the biosensor molecule when placed under the control of appropriate regulatory sequences. For example, the boundaries of a coding region are determined by a start codon at the 5′ (amino) terminus and a translation stop codon at the 3′ (carboxy) terminus. A gene can include, but is not limited to, cDNA from prokaryotic or eukaryotic mRNA, genomic DNA sequences from prokaryotic or eukaryotic DNA, and synthetic DNA sequences. A transcription termination sequence will usually be located 3′ to the gene sequence.

The expression vector includes, at the least, a promoter. Transcription of DNA is dependent upon the presence of a promoter which is a DNA sequence that directs the binding of RNA polymerase and thereby promotes mRNA synthesis. The DNA sequences of eukaryotic promoters differ from those of prokaryotic promoters. Furthermore, eukaryotic promoters and accompanying genetic signals may not be recognized in or may not function in a prokaryotic system and prokaryotic promoters are not recognized and do not function in eukaryotic cells. Eukaryotic promoters typically lie upstream of the gene and can have regulatory elements several kilobases away from the transcriptional start site. In eukaryotes, the transcriptional complex can cause the DNA to bend back on itself, which allows for placement of regulatory sequences far from the actual site of transcription. Many eukaryotic promoters, between 10 and 20% of all genes, contain a TATA box (sequence TATAAA), which in turn binds a TATA binding protein that assists in the formation of the RNA polymerase transcriptional complex. The TATA box typically lies very close to the transcriptional start site, often within 50 bases (Gershenzon et al., Bioinformatics 21:1295-300 (2005) and Smale et al., Annual Review of Biochemistry 72:449-479 (2003), which are hereby incorporated by reference in their entirety). Eukaryotic promoter regulatory sequences typically bind proteins called transcription factors which are involved in the formation of the transcriptional complex. An example is the E-box (sequence CACGTG), which binds transcription factors in the basic-helix-loop-helix (bHLH) family (e.g., BMAL1-Clock, cMyc).

Promoters vary in their “strength” (i.e., their ability to promote transcription). For the purposes of expressing a cloned gene, it is generally desirable to use strong promoters in order to obtain a high level of transcription and, hence, expression of the gene. Depending upon the host cell system utilized, any one of a number of suitable promoters may be used. For instance, when cloning in Escherichia coli, its bacteriophages, or plasmids, promoters such as the T7 phage promoter, lac promoter, trp promoter, recA promoter, ribosomal RNA promoter, the P_{R} and P_{L} promoters of coliphage lambda and others, including but not limited to, lacUV5, ompF, bla, lpp, and the like, may be used to direct high levels of transcription of adjacent DNA segments. Additionally, a hybrid trp-lacUV5 (tac) promoter or other E. coli promoters produced by recombinant DNA or other synthetic DNA techniques may be used to provide for transcription of the inserted gene. Bacterial host cell strains and expression vectors may be chosen which inhibit the action of the promoter unless specifically induced. In certain operations, the addition of specific inducers is necessary for efficient transcription of the inserted DNA. For example, the lac operon is induced by the addition of lactose or IPTG (isopropylthio-beta-D-galactoside). A variety of other operons, such as trp, pro, etc., are under different controls.

A constitutive promoter is a promoter that directs expression of a gene throughout the development and life of an organism. An inducible promoter is a promoter that is capable of directly or indirectly activating transcription of one or more DNA sequences or genes in response to an inducer. In the absence of an inducer, the DNA sequences or genes will not be transcribed.

Additional control elements, such as an enhancer and/or a transcription termination signal, may also be included in the expression vector. The term “control elements” refers collectively to promoter regions, polyadenylation signals, transcription termination sequences, upstream regulatory domains, origins of replication, internal ribosome entry sites, enhancers, splice junctions, and the like, which collectively provide for the replication, transcription, post-transcriptional processing, and translation of a coding sequence in a recipient cell. Not all of these control elements need always be present, so long as the selected coding sequence is capable of being replicated, transcribed, and translated in an appropriate host cell.

By “enhancer element” it is meant a nucleic acid sequence that, when positioned proximate to a promoter, confers increased transcription activity relative to the transcription activity resulting from the promoter in the absence of the enhancer domain. Hence, an “enhancer” includes a polynucleotide sequence that enhances transcription of a gene or coding sequence to which it is operably linked. A large number of enhancers, from a variety of different sources, are well known in the art. A number of polynucleotides which have promoter sequences (such as the commonly used CMV promoter) also have enhancer sequences. The enhancer or the promoter can be tissue specific. Tissue-specific enhancers (and promoters) help direct gene expression in a particular cell type and do not direct gene expression in all tissues or all cell types. Tissue-specific enhancers or promoters may be naturally occurring or non-naturally occurring. One skilled in the art will recognize that the synthesis of non-naturally occurring enhancers or promoters can be performed using standard oligonucleotide synthesis techniques.

The vector of choice, promoter, and an appropriate 3′ regulatory region can be ligated together to produce the expression vector of the present invention using well known molecular cloning techniques as described in Sambrook et al., Molecular Cloning: A Laboratory Manual, Second Edition, Cold Spring Harbor Press, NY (1989), and Ausubel, F. M. et al. Current Protocols in Molecular Biology, New York, N.Y: John Wiley & Sons (1989), each of which is hereby incorporated by reference in its entirety.

Pursuant to the method of the present invention, the expression vector encoding the biosensor molecule is introduced locally into in vivo cells of a subject under conditions effective to express the biosensor molecule in the cells. In one embodiment, expression of the biosensor molecule is localized to a particular cell, group of cells, tissue, or region of the subject into which the cells carrying the expression vector have been introduced or are known to reside. Thus, the present invention relies on manipulation of the genetic code in in vivo cells. By “in vivo” it is meant that the biosensor molecule is expressed in cells which exist in tissue of a subject or can be introduced and maintained in tissue of a subject. Upon introduction of the expression vector into a cell, the genetic code of the cell is modified such that it genetically expresses the biosensor molecule. In one embodiment, cells that express the biosensor molecule of the present invention are sustained by and derive nutrients from the subject.

The expression vector encoding the biosensor molecule can be introduced into a variety of cells and tissues. In one embodiment, the cells are epithelial cells. By “epithelial cell” or “epithelial tissue” it is meant a cell or group of cells derived from the epithelium. The term includes epithelical cells both in vitro and in vivo. Thus, for example, the expression vector encoding the biosensor molecule may be introduced into, e.g., epithelial cells in culture and then the epithelial cells are introduced into a local epithelial tissue of the subject. According to this embodiment, the cells may or may not be cells that originated from the subject. Alternatively, the expression vector may be introduced into, e.g., epithelial cells that reside in the subject. For purposes of the present invention, the epithelium is a tissue composed of cells that line the cavities and surfaces of structures throughout the body. Many glands are also formed from epithelial tissue. Epithelial cells include both differentiated and nondifferentiated epithelial cells.

The tissue or cells which are used in the present invention can be a xenogeneic relative to the intended subject. Such tissue can be transplanted into the subject after the expression vector is introduced into them. The cells may be isolated from, e.g., cardiac tissue, skeletal muscle tissue, bone marrow, or umbilical cord blood. Methods of culturing cells and/or methods of inducing differentiation of cells are known in the art. For example, methods to induce differentiation of embryonic stem cells, bone marrow cells, or hematopoietic stem cells to cardiac cells, are described in U.S. patent application Ser. No. 10/722,115, which is hereby incorporated by reference in its entirety.

The expression vectors carrying the nucleic acid molecule encoding the biosensor molecule of the present invention can be introduced locally via any procedure currently known or to be discovered including, for example, transdermal, intramuscular, subcutaneous, buccal, rectal, intravenous, or intracoronary administration (U.S. Pat. No. 5,328,470 to Nabel et al., which is hereby incorporated by reference in its entirety) or by stereotactic injection (see e.g., Chen et al. Proc. Nat'l. Acad. Sci. USA 91:3054-3057 (1994), which is hereby incorporated by reference in its entirety). In one embodiment, introducing is carried out transdermally or intradermally. The expression vector is preferably introduced in a manner such that the recipient tissue or cells are in direct communication with the target physiological molecule or analyte, e.g., if the target is a blood-borne molecule or analyte then the biosensor molecules are delivered to cells or tissue that are in direct contact with the endogenous blood supply.

The expression vector of the present invention can be introduced into cells by a variety of well-known techniques such as vector-mediated gene transfer (by, e.g., viral infection/transfection, or various other protein-based or lipid-based gene delivery complexes, gold mediated transfer) as well as techniques facilitating the delivery of “naked” polynucleotides (such as electroporation, “gene gun” delivery and various other techniques used for the introduction of polynucleotides). Another method of delivering the expression vector to the targeted tissue or cells is by using MEMS microneedle arrays (Xie et al., Nanomedicine: Nanotechnology, Biology, and Medicine 1:184-190 (2005); Davis et al., IEEE Transactions on Biomedical Engineering 52:909 (2005); Mukerjee et al., Sensors and Actuators 114:267-275 (2004), which are hereby incorporated by reference in their entirety)

The introduced expression vector (or polynucleotide encoding the biosensor molecule) may be stably or transiently maintained in the host cell. Stable maintenance typically requires that the introduced polynucleotide either contain an origin of replication compatible with the host cell or integrates into a replicon of the host cell such as an extrachromosomal replicon (e.g., a plasmid) or a nuclear or mitochondrial chromosome.

A number of vectors are known to be capable of mediating transfer of genes to mammalian cells. Transduction denotes the delivery of a polynucleotide to a recipient cell either in vivo or in vitro, via a viral vector and preferably via a replication-defective viral vector, such as via a recombinant AAV. One approach involves microinjection, where DNA is injected directly into the nucleus of cells through fine glass needles. Alternatively, the nucleic acid molecule can be introduced using dextran incubation, in which DNA is incubated with an inert carbohydrate polymer (dextran) to which a positively charged chemical group (e.g., diethylaminoethyl (“DEAE”)) has been coupled. The DNA sticks to the DEAE-dextran via its negatively charged phosphate groups. These large DNA-containing particles stick in turn to the surfaces of cells, which are thought to take them in by a process known as endocytosis. Some of the DNA evades destruction in the cytoplasm of the cell and escapes to the nucleus, where it can be transcribed into RNA like any other gene in the cell.

In another embodiment, the expression vector is introduced using calcium phosphate coprecipitation, where the target cells efficiently take in DNA in the form of a precipitate with calcium phosphate.

Electroporation is another means for achieving cellular transfection. Using this method, cells are placed in a solution containing DNA and subjected to a brief electrical pulse that causes holes to open transiently in their membranes. DNA enters through the holes directly into the cytoplasm, bypassing the endocytotic vesicles through which they pass in the DEAE-dextran and calcium phosphate procedures (passage through these vesicles may sometimes destroy or damage DNA).

Liposomal mediated transformation is yet another suitable approach for transfecting cells with DNA. Using this method the nucleic acid molecule is incorporated into an artificial lipid vesicle, a liposome, which fuses with the cell membrane, delivering its contents directly into the cytoplasm of the target cell.

Delivery of a nucleic acid molecule can also be achieved using biolistic transformation, in which DNA is absorbed to the surface of gold particles and fired into cells under high pressure using a ballistic device.

Also, viral-mediated transformation, using any of the viral vectors described herein, is another approach for introducing a nucleic acid molecule into a target cell.

The number of cells that are transfected by the administered expression vector can vary. The amount of tissue or cells that are transfected may also vary depending on the mode of delivery, uptake of expression vector by cells, height, weight, gender, age, and condition of the subject.

In one embodiment the expression vector can include the vector in an acceptable diluent, or can comprise a slow release matrix in which the gene delivery vehicle is imbedded.

The expression vector of the present invention may be introduced in various locations within a subject. For example, it may be introduced in a subcutaneous pocket anywhere on the body, or in any tissue or organ of the body. Locations for introduction of a biosensor molecule include, for example, any part of the major organ systems such as circulatory, digestive, endocannabinoid, endocrine, integumentary, immune, lymphatic, musculoskeletal, nervous, reproductive, respiratory, urinary, and vestibular system. The tissues and cells that can be used for the present invention include tissues and cells which are part of the nervous tissue, muscle tissue, connective tissue, or epithelial tissue. In certain embodiments brain, heart, skin, mouth, vascular lumen, bones, eyes, and lungs can be the targeted tissue or cells.

Transgenic tissue or cells comprising the expression vector of the present invention, can also be transplanted or implanted at these locations. For example, prior to implant into the subject the cells are made transgenic by introducing the expression vector of the present invention. When these transgenic cells are implanted into the subject, the expression vector expresses the biosensor molecule and thus confers the ability to detect analytes or physiological molecules. In one embodiment, the transgenic tissue or cells which are to be implanted are present in and/or on a biocompatible matrix, e.g., a collagen-based matrix, or on the surface of an implantable device such as an electrical lead, e.g., one having a biocompatible matrix applied thereto.

Once expressed in the cell locally in vivo in the subject, the biosensor molecule is used to repeatedly and chronically track changes in molecules or analytes found in tissue or physiological fluid of the subject. Thus, for example, the present invention achieves the advantage of minimizing infrequent and inconvenient monitoring of analytes by e.g., benchtop analysis of blood samples. In one embodiment of the present invention, signal from the expressed biosensor molecule correlates to relative abundance of an analyte in the subject. Thus, for example, an important medical use of the present invention is for monitoring the concentration of an analyte in a subject. In one embodiment, the cells expressing the biosensor molecule can be replenished by reintroducing them locally in vivo in the subject. For example, it may be desirable to re-introduce the expression vector into localized cells or tissue of the subject weekly, monthly, every six months, or yearly, depending upon the specific embodiment employed.

Examples of physiological molecules or analytes which can be detected using the method of the present invention include, but are not limited to, molecules found in physiological fluids such as blood, seminal fluid, cerebrospinal fluid, lymphatic fluid. The molecules or analytes include, but are not limited to, glucose, insulin, endocrine, paracrine or autocrine hormones, biomarkers, pathogens, drugs, or toxins. In one embodiment, the analyte is a disease-related biomarker. Thus, the invention can involve monitoring biomarkers related to disease (e.g. cardiac disease, cancer, Alzheimer's, etc).

In another embodiment, the analyte is a pharmaceutical drug administered to a subject. For example, it might be useful to measure the dose of a pharmaceutical drug in target tissues (even internal organs if the sensing moiety was selected to provide adequate depth penetration in such tissue). A “drug” as used herein is an agent which, in an effective amount, has a prophylactic or therapeutic effect.

Alternatively, the method of the present invention is employed to detect an analyte associated with an indication of substance abuse in the subject.

In one embodiment, the analyte is glucose and the analyte binding domain of the biosensor molecule is, e.g., a glucose/galactose-binding protein (“GGBP”) domain. Glucose detection is currently done (clinically) with a blood sample. This method is painful for the patient and does not allow continuous monitoring. There has been significant research on implanted glucose sensors, most based on glucose oxidase. These suffer from stability issues, plus significant issues with implant encapsulation and rejection. The approach of the present invention allows for a “non-invasive” assessment of glucose levels in a continuous format.

GGBP is a type of protein naturally found in the periplasmic compartment of bacteria. These proteins are naturally involved in chemotaxis and transport of small molecules (e.g., sugars, amino acids, and small peptides) into the cytoplasm. GGBP is a single chain protein consisting of two globular/domains that are connected by three strands to form a hinge. The binding site is located in the cleft between the two domains. When glucose enters the binding site, GGBP undergoes a conformational change, centered at the hinge, which brings the two domains together and entraps glucose in the binding site. X-ray crystallographic structures have been determined for the closed form of GGBP from E. coli (Vyas et al., Science 242:1290-1295 (1998), which is hereby incorporated by reference in its entirety) and S. typhimurium (Mowbray et al., Cole Receptor 1:41-54 (1990), which is hereby incorporated by reference in its entirety). The wild type E. coli GGBP DNA and amino acid sequence are accessible with the Protein Databank Accession Number D90885 (genomic clone) and Accession Number 23052 (amino acid sequence), which are hereby incorporated by reference in their entirety.

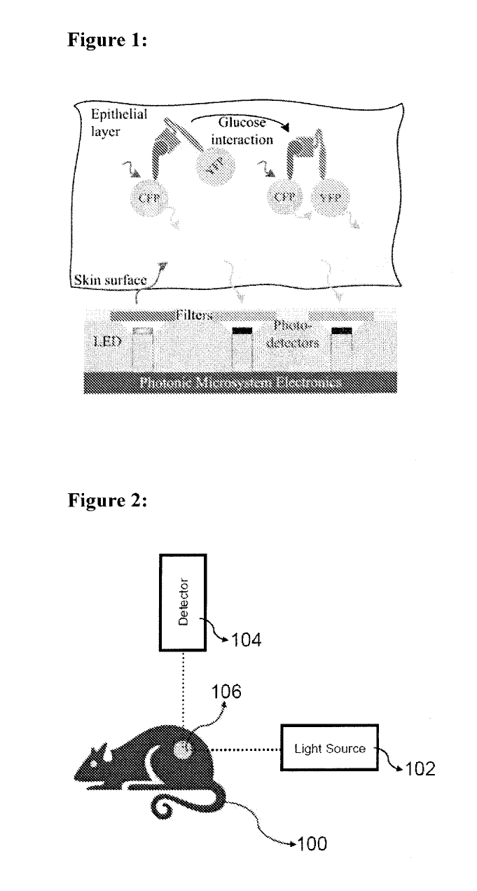

The general concept for a glucose biosensor molecule is illustrated in FIG. 1. In particular, FRET pairs of proteins are tuned for conformational change in response to binding of the glucose analyte. Epithelial layer cells are transfected with vectors to express the biosensor protein molecules. The expressed biosensor molecule directly samples the interstitial fluid with an external photonic microsystem monitoring the spectral characteristics of the FRET pair. These spectral characteristics allow determination of the conformation of the FRET protein pair which is correlated to glucose concentration. Since the protein is expressed locally in the subject by the cells, there is continous renewal of the sensing modality reducing issues associated with photo-bleaching in the assay. The present invention will significantly improve the treatment efficacy for diabetic patients on an insulin regimen by more frequent measurement of blood glucose levels; a frequency which is limited by the associated blood sampling. The present invention allows continuous glucose detection without a need for an implanted device. However, in certain embodiments there could be an implanted device that is in communication with an external device such that the external device can non-invasively detect signals from the implanted device and the implanted device detects signals from the biosensor molecules of the present invention and conveys the information to the external device.

GGBP can be mutated to alter glucose binding affinity. Examplary mutations may include proteins from bacteria containing an amino acid(s) which has been substituted for, deleted from, or added to the amino acid(s) present in naturally occurring protein. Exemplary mutations of binding proteins include the addition or substitution of cysteine groups and/or non-naturally occurring amino acids (Turcatti et al., J. Bio. Chem. 271:19991-19998 (1996), which is hereby incorporated by reference in its entirety) and replacement of substantially non-reactive amino acids with reactive amino acids to provide for covalent attachment to surfaces. The mutated binding protein or GGBP is capable of following the kinetics of biological reactions involving glucose.

Mutations introduced in the biosensor molecule of the present invention may serve one or more of several purposes. For example, a naturally occurring protein may be mutated in order to change the long-term stability of the protein; to conjugate, bind, couple, or otherwise associate the protein to a particular encapsulation matrix polymer or surface; adjust its binding constant with respect to a particular analyte; and combinations thereof.

The analyte and mutated protein can act as binding partners. The term “associates” or “binds” as used herein refers to binding partners having a relative binding constant (Kd) sufficiently strong to allow detection of binding to the protein by a detection means. The Kd may be calculated as the concentration of free analyte at which half the protein is bound, or visa versa.

Thus, in one embodiment, the signal from the signal domain of the biosensor molecule is a FRET between the first and second fluorescent protein domains. FRET probe technology is well advanced and used extensively for molecular level studies, primarily in vitro. There are existing FRET probe pairs for glucose binding protein. No work has been reported in the literature for in vivo use of this for glucose monitoring in mammals.

Pursuant to the method of the present invention, a signal from the expressed biosensor molecule is detected by a non-invasive means. Thus, for example, a fluorescent microscope with FRET detection capability can be used for optical evaluation. In one embodiment, blood glucose levels can be monitored with a commercial meter and compared to FRET output across a range of levels driven by either intraperitoneal or intravenous glucose injections. For example, a FRET-based in vivo glucose detection which uses genetic manipulations for expression of the fluorescent sensor proteins can be employed. Starting with a cyan/yellow FRET pair coupled to a glucose binding protein sensory domain, DNA constructs can be prepared and transiently expressed in cultured cells. Assays can be performed to demonstrate glucose concentration sensitivity using a fluorescent microscope. These constructs can be packaged in a lentivirus and injected subcutaneously or intradermally in a subject for localized FRET expression. Using the fluorescent microscope, FRET measurements can be performed in vivo. Blood glucose levels will be monitored using blood samples and standard laboratory techniques. Photo-bleaching and rate of renewal can also be evaluated using the techniques described herein.

The method of the present invention detects the presence or amount of an analyte in a subject by non-invasive means. By “non-invasive” it is meant that no break in the skin is created and there is no contact with the mucosa, or skin break, or internal body cavity beyond a natural or artificial body orifice. For example, methods like pulse-taking, the auscultation of heart sounds and lung sounds (using the stethoscope), temperature examination (using thermometers), respiratory examination, peripheral vascular examination, oral examination, abdominal examination, external percussion and palpation, blood pressure measurement (using the sphygmomanometer), change in body volumes (using plethysmograph), audiometry, eye examination are all non-invasive procedures.

The methods of the present invention include transmitting to an external system from in vivo cells in the subject, a signal corresponding to the presence and/or amount of one or more detected physiological molecules or analytes. The transgenic tissue or cells are capable of being coupled to a detector, by non-invasive means, adapted to detect a signal from the transgenic tissue or cells. The transgenic tissue or cells may be augmented with another expression cassette comprising a transcriptional regulatory element operably linked to an open reading frame encoding a protein which is capable of associating with the cell membrane and binding the one or more physiological molecules, which binding alters the amount and/or activity of one or more intracellular second messenger molecules in the transgenic cells and which one or more intracellular second messenger molecules in turn modulate the activity of one or more ion channels, which modulation is detected by the detector.

This signal from the biosensor molecule can be detected by various methods known in the art. These methods include non-invasive methods like optical measurements such as UV, IR, bioluminescence measurements or imaging, fluorescence measurements or imaging, dermatoscopy, diffuse optical tomography, use of gamma camera and other scintillographical methods, such as positron emission tomography and single-photon emission tomography, using radioactive tracers in the body, computed tomography, gene expression imaging, infrared imaging of the body, magnetic resonance elastography, magnetic resonance imaging using external magnetic fields, magnetic resonance spectroscopy, optical coherence tomography, posturography, radiography, fluoroscopy and computed tomography, using X-rays, ultrasonography and echocardiography using ultrasound waves for imaging.

In one embodiment, the detecting is carried out with a fluorometer. For example, a patch of epithelial cells (skin) of a subject is transfected to express a FRET pair that responds to glucose binding protein. An external light source and a fluorometer is placed against the skin to do a non-invasive measurement of glucose using the fluorescence from the FRET pair. Since the biosensor protein is being expressed by the epithelial cells, it will be constantly renewed, alleviating issues with photobleaching.

Pursuant to the method of the present invention, subjects are genetically modified, locally, to express a biosensor molecule. A suitable subject for carrying out the method of the present invention can be any plant and/or animal. Preferable animals include mammals. By “mammal” it is meant any member of the class Mammalia including, without limitation, humans and nonhuman primates such as chimpanzees and other apes and monkey species; farm animals such as cattle, sheep, pigs, goats, and horses; domestic mammals such as dogs and cats; laboratory animals including rodents such as mice, rats, rabbits, guinea pigs, and the like. Preferably, the subject is a human.

### EXAMPLES

### Example 1

### DNA Design and Lentivirus Vector Construction

A glucose sensing probe was generated using fluorescence resonance energy transfer (FRET). The glucose sensor was made following the procedure described in Fehr et al., J. Biol. Chem. 278:19127-33 (2003), which is hereby incorporated by reference in its entirety. Briefly, the cDNA sequence for glucose/galactose-binding protein (GGBP) from Haemophilus influenzae was identified in the gene bank (Genbank ID: YP_{—}248529.1). The GGBP protein has the following amino acid sequence (SEQ ID NO: 1):

```
    1 mmyttlsihi nlpnrsvimk ktavlstvaf aialgsasas faadnrigvt
  51 iykyddnfms lmrkeidkea kvvggikllm ndsqnaqsiq ndqvdillsk
101 gvkalainlv dpaaaptiig kaksdnipvv ffnkdpgaka igsyeqayyv
151 gtdpkesgli qgdliakqwk anpaldlnkd gkiqfvllkg epghpdaevr
201 tkyvveelna kgiqteqlfi dtgmwdaama kdkvdawlss skandievii
251 snndgmalga leatkahgkk lpifgvdalp ealqliskge lagtvlndsv
301 nqgkavvqls nnlaqgksat egtkwelkdr vvripyvgvd kdnlgdflk
```

The GGBP sequence was mutated in order to effectively change its glucose binding affinity to more physiologically relevant glucose levels, as described in Fehr et al., J. Biol. Chem. 278:19127-33 (2003), which is hereby incorporated by reference in its entirety. This new construct was then flanked by fluorescent proteins. At the N-terminus, a yellow fluorescent protein (YFP) was fused and at the C-terminus, cyan fluorescent protein (CYP) was fused. The chimeric gene was inserted into pRSET (Invitrogen) or pcDNA3.1 (Invitrogen) and transferred to E. coli BL21(DE3) Gold (Stratagene) and COS-7 cells.

The excitation and emission spectra of the two fluorescent proteins enabled FRET to occur when in close association. Glucose binding caused a conformational change in GGBP which separated the fluorescent proteins to reduce FRET. This fusion construct was made with PacI restriction enzyme sites between the fluorescent proteins and GGBP, enabling the fluorescent proteins to be easily changed, e.g., if there is a need to get better sensing intensities. The construct was further codon-optimized for translation within rodents. The entire cDNA was then subcloned into a standard lentivirus backbone, which uses the chicken beta-actin promoter with a CMV enhancer to drive transcription of the cDNA. This vector has an excellent longterm expression both in vitro and in vivo. This vector was used for lentivirus production.

### Example 2

### Lentivirus Production

Briefly, 293FT cells were transiently transfected with 1) one shuttle vector plasmids, 2) a packaging plasmid encoding the HIV-1 Gag and Pol proteins, 3) an envelope plasmid encoding the vesicular stomatitis virus glycoprotein (VSV-G) to confer broad tropism and 4) a plasmid encoding the Rev post-transcriptional regulator that is required for Gag/Pol expression. HEK293FT cells were transfected using CaPO₄ precipitation with a ratio of the above vectors being 1.8:1.5:1:1 ratio. Cells were re-fed 24 hours later with fresh DMEM+10% NCS. Packaged virus was collected 48 hours later; virus containing cell media was collected, spun for 5 minutes at 1000 g, passed through a 0.45 μm millipore filter and ultracentrifuged over a 20% sucrose cushion at 25,900 rpm in a SW-32 rotor for 2 hours at 4° C. When finished, the supernatant was aspirated, and the virus-containing pellet was resuspended in 40 μl phosphate-buffered saline (PBS) containing 1 mg/ml Rat albumin.

### Prophetic Example 3

### Cell Culture and In Vitro Characterization

HEK 293 cells will be cultured at 37° C., 5% CO₂ in DMEM (or Minimum Essential Medium) supplemented with 100 units/ml penicillin G sodium, 100 mg/ml streptomycin, 4 mM L-glutamine, and 10% fetal bovine serum. Cells will be transfected with the lentivirus for expression of the FRET construct in the cell cytosol. FRET expression will be characterized 30-40 hours post-transfection using a fluorescent microscope equipped with a CFP/YFP filter set. Dual emission intensity ratios will be recorded and ratio changes calculated in response to perfusates of different glucose concentrations. A constant flow perfusion system will be utilized allowing flow of glucose-free culture medium, and glucose containing medium with concentrations ranging from 100 μM to 40 mM. FRET ratio versus glucose concentration will be quantified with high and low saturation ranges determined.

To evaluate photobleaching and rate of renewal, repeated measures will be done on cultured cells with a stable media environment. A region of the culture dish will be marked prior to culture to allow repeated measures in the same population of cells. Optical measurements will continue until loss of FRET signal from photobleaching. The culture will then be allowed to recover with subsequent FRET measurements from the same population of cells. The recovery period will be varied to determine the rate of renewal and potential challenges associated with photobleaching in this application.

These in vitro characterization experiments can also be used to characterize and adjust properties of fluorescent proteins and FRET pairs such as photonic efficiency, excitation and emission wavelengths, and quantum yields. For example, fluorescent proteins can be mutated to adjust their emission or excitation wavelengths so that the emitted or excitation light can travel deeper in and out of the tissue. This is generally achieved by shifting the wavelength, e.g., to red regions of the spectrum.

### Prophetic Example 4

### Animal Studies and In Vivo Demonstration

To evaluate the potential for in vivo monitoring of glucose concentrations, CBA/CaJ mice (The Jackson Laboratory, Maine) (shown in FIG. 2 as 100) will undergo intradermal injections of lentivirus for expression of FRET within the epithelial cell layer of the mice (shown in FIG. 2 as 106). Animals will be deeply anesthetized with a mixture of ketamine/xylazine (120 and 10 mg/kg body weight, respectively, intraperitoneal injection) with supplementary doses (1/3 of the initial dose) administered as needed. Parameters such as foot or tail pinch, palpebral reflex and respiratory rate will be monitored to indicate the need for supplemental doses. The left dorsal posterior surface of the back will be injected with intradermal injection of lentivirus after shaving and cleaning. Following injection, the animal will be kept in a holding cage under a small heat lamp (temperature monitored in the cage with a thermometer) until it awakens and moves about normally. The animal will be observed for any signs of distress, including excessive scratching at the injection site, or bleeding. After full recovery the animal is then returned to its cage to be returned to the Vivarium. Food will be withheld for 12 hours prior to FRET assessment and blood glucose measurement.

48 hours post injection the animal will be returned to the lab for FRET assessment using the same microscopic setup described for the in vitro characterization. In one embodiment this microscopic setup will include a light source (shown in FIG. 2 as 102) and a detector (shown in FIG. 2 as 104). Animals will be anesthetized as described above and placed on a heated pad on the microscope stage. FRET ratios will be determined at four locations at the lentivirus injection site. A blood sample will be obtained from the tail following procedures described by Hoff, Lab Animal 29(10):47-53 (2000), which is hereby incorporated by reference in its entirety. A 2 mm distal section of the mouse's sterilized tail is snipped using a scapel and gently squeezed to obtain two drops of blood, the first of which is discarded. Blood glucose is measured from the second drop using the Johnson and Johnson's One Touch Ultra Blood Glucose Monitoring System (Johnson and Johnson, New Brunswick, N.J.) which requires only 1 μl of blood and provides results in 5 seconds. This testing procedure is described by Vasilyeva (Vasilyeva et al., Hearing Research 249:44-53 (2009), which is hereby incorporated by reference in its entirety).

To modulate blood glucose levels, glucose will be administered either through intraperitoneal injection or intravenous injection. Both FRET and glucose measurements will be repeated at 15, 30, 60, and 120 minutes post glucose injection. FRET ratio will be analyzed as a function of blood glucose level.

The photobleaching experiments described for cultured cells will be repeated in vivo following a similar approach; repeated FRET measurement with subsequent photobleaching, variable recovery period, and repeated FRET measurement at the same location.

### Prophetic Example 5

### Microsystem Requirements

Translational results in humans will ultimately require a small measurement system which can be worn by the patient. This system will require an excitation source, filtering, and two photodetectors as illustrated in FIG. 1. Using microscope specifications in combination with measurements of FRET expression with modulated excitation source intensity, microsystem photonic component requirements will be defined. First principle analysis of FRET and associated bleedthrough will be used to define an in vivo calibration procedure which would allow clinical determination of blood glucose based on the expressed nanosensor and the photonic microsystem.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

What is claimed:

1. An in vivo method of monitoring an analyte in a subject, said method comprising:
providing an expression vector that encodes a biosensor molecule, the biosensor molecule comprising an analyte binding domain and a signal domain, wherein the biosensor molecule produces a signal from the signal domain upon binding of the analyte by the analyte binding domain, the signal being detectable by a non-invasive means;
introducing the expression vector locally into in vivo cells of a subject under conditions effective to express the biosensor molecule in the cells; and
detecting, by a non-invasive means, the signal from the expressed biosensor molecule, thereby monitoring the analyte in the subject in vivo.

2. The method according to claim 1, wherein signal strength correlates to relative abundance of the analyte in the cell.

3. The method according to claim 1, wherein the signal domain comprises a fluorescent protein domain.

4. The method according to claim 3, wherein the biosensor molecule further comprises a second signal domain.

5. The method according to claim 4, wherein the second signal domain comprises a fluorescent protein domain.

6. The method according to claim 5, wherein the first and second fluorescent protein domains are separated by the analyte binding domain.

7. The method according to claim 6, wherein the second fluorescent protein domain is different from the first fluorescent protein domain.

8. The method according to claim 7, wherein the signal is a fluorescent resonance energy transfer (FRET) between the first and second fluorescent protein domains.

9. The method according to claim 8, wherein said binding of the analyte by the analyte binding domain results in a conformational change in the analyte binding domain.

10. The method according to claim 9, wherein the analyte binding domain is a glucose/galactose-binding protein domain and the analyte is glucose.

11. The method according to claim 10, wherein the glucose/galactose-binding protein is mutated to alter glucose binding affinity.

12. The method according to claim 8, wherein said detecting is carried out with a fluorometer.

13. The method according to claim 1, wherein the cells are epithelial cells.

14. The method according to claim 13, wherein said introducing is carried out transdermally or intradermally.

15. The method according to claim 1, wherein the subject is a human subject.

16. The method according to claim 1, wherein said introducing is carried out by transfection.

17. The method according to claim 1, wherein the analyte is a disease-related biomarker.

18. The method according to claim 1, wherein the analyte is a pharmaceutical drug administered to the subject.
